(19) United States
(12) Patent Application Publication
(10) Pub. No.: US 2004/0120975 A1
(43) Pub. Date: Jun. 24, 2004

(54) Biliquid foam emulsions of water and hydrofluoroether as cosmetic carriers

(76) Inventors: Konstantinos M. Lahanas, Paramus, NJ (US); Eugene Puchalski JR., Jersey City, NJ (US); Amit R. Shah, Commack, NY (US); Philip Ernest Guffogg, Surrey (GB)

Correspondence Address: NOTARO AND MICHALOS, 100 DUTCH HILL ROAD, SUITE 110, ORANGEBURG, NY, 10962-2100, US

(21) Appl. No.: 10/328,308

(22) Filed: Dec. 23, 2002

(51) Int. Cl. A61K 7/00

(52) U.S. Cl. 424/401

(57) ABSTRACT

A perfluorinated oil-in-water biliquid foam is provided as a vehicle for delivering cosmetics and active agents while producing a refreshing and unique contact with the skin, and enhancing the aroma of fragrances within the foam. The biliquid foam contains an oil phase perfluorinated solvent, surfactants, co-solvents, and oil-based ingredients such as aromatic extracts, and a water phase with anionic or hydrophilic surfactants. The biliquid foam may be made by gradually increasing addition of the oil phase to the water phase under moderate mixing conditions. Various other auxillary cosmetic and non-cosmetic components such as vitamins, anti-oxidants, stabilizers, thickeners, dyes, moisturizers, and preservatives may be added to the biliquid foam as well.

### FIELD AND BACKGROUND OF THE INVENTION

The present invention relates generally to the field of cosmetics and in particular to a new and useful biliquid foam emulsion of a high concentration of perfluorinated oil in water which carries fragrances and other active agents.

Cosmetics have a variety of applications, such as providing a fragrance or preventing and/or correcting skin damage from harmful environmental conditions via an active agent such as an anti-oxidant. Cosmetics can be applied to the skin in many ways such as by pump spray or topical application.

However, a fragrance or active agent cannot be conveyed to the skin, without a carrier. Cosmetics exist in many forms including lotions, creams, gels, and perfumes. In perfumes, for example, the fragrance is normally conveyed via a solvent. Many solvents are not adequate, however. Ethanol, for example, is commonly used as a carrier for conveying a fragrance from perfumes, but the odor of alcohol often interferes with the natural fragrance of the perfume. Generally, fragrances can also be difficult to dissolve in fluids.

Additionally, many cosmetics cause skin irritation, dryness, rashes, blotches or other more severe skin damage such as oxidation or DNA crosslink damage. Carriers must be biologically and chemically non-reactive and mild to the skin, particularly with sensitive skin.

Cosmetics must not only be non-toxic to the skin, but also non-toxic to the environment. Although CFC's have virtually been abolished, they had caused much harm to the ozone layer and the environment. A cosmetic carrier is needed that has low atmospheric lifetime and is biodegradable.

Finally, there is a known tendency for emulsifiers in cosmetics to deactivate preservatives by an absorption process, resulting in the diminution of the amount of free unabsorbed active agent. This tendency has implications for the effectiveness of the preservation of the cosmetic as well as anti-microbial agents.

According to the present invention, a new perfluorinated oil-in-water dispersion is provided as a cosmetic carrier, and in particular, a fragrance carrier, that does not interfere with the fragrance that it carries, is mild and non-toxic to the skin and the environment, and does not destroy preservatives. In fragrance applications in particular, the perfluorinated oil-in-water dispersion of the invention enhances the properties of the fragrant agent to provide a stronger fragrance. Although the perfluorinated oil-in-water dispersion is most useful in the field of cosmetic fragrances, it is also suitable for conveying chemically active and bioactive agents.

Typically, perfluorinated compounds are extremely difficult to emulsify in water. They are insoluble in water and insoluble in most oils as well. However, the present invention teaches the incorporation of 10-90% HFE in water by dropwise addition and stirring of HFE/oil surfactant mixture into water.

In part, the incorporation of such a high concentration of HFE in water is accomplished by the formation of a biliquid foam, which is a type of oil-in-water dispersion, resembling colloidal gas foams and emulsions, in which droplets of oil are suspended in an aqueous environment and prevented from coalescing by a film of surfactant. However, the surfactant film acts as a third phase distinct from the water and oil phases of the biliquid foam.

The extremely high concentration of perfluorinated oil compound that is dispersed in water to function as a carrier for fragrances and active agents, and in particular, to amplify the effect of fragrances upon skin contact, is not known or practiced in the art.

For example, published European patent application EP 1 029 527 to Daiken Industries teaches a cosmetic preparation where a hydrofluroether (CₙHₘFₗ—O—CₓH_{y}F_{z}) may be incorporated into a cosmetic alone or in a mixture with fluorine containing oils. A preferred embodiment of hydrofluoroether comprises one perfluorinated alkane branch and one aliphatic alkoxy branch that does not contain fluorine. This preferred embodiment of hydrofluoroether may be best represented by the general chemical formula CₙF₂ₙ₊₁—O—CₓH₂ₓ₊₁. The amount of HFE that may be present alone in the cosmetic at least as disclosed in the reference, ranges from 1-99% by weight, and in particular 10-99% by weight, based on the cosmetic.

The Daikin patent application further discloses HFE dissolved in perfluoropolyether, a skin moisturizer. The Daikin patent teaches non-aqueous lotion mixture of 50% by weight perfluoropolyether and 50% by weight HFE (C₄F₉OCH₃). Upon application to skin, the HFE evaporates, and a single coat of the cosmetic perfluropolyether remains. Several other embodiments of the disclosure involve a replacement of perfluoropolyether with a mixture of fluorine-containing oils or fluorine compound-treated powders.

Daikin discloses HFE's ability to dissolve fluorine-containing oils, and focuses on an application of HFE to fluorine-containing cosmetic lotions. Although according to Daiken, HFE can be incorporated into a cosmetic alone at 99% by weight, or into a non-aqueous lotion at 50% by weight, Daikin does not teach the dispersion of HFE in water at all. Even though perfumes are disclosed as a possible cosmetic, there is no description of the effect of HFE on fragrances.

Published PCT application WO 99/26600 to Archimex discloses a cosmetic composition comprising an aromatic cosmetic dissolved in a perfluorinated HFE such as methoxynonafluorobutane (C₄F₉OCH₃) or any of its isomers. A co-solvent may be included to assist in the incorporation of the fragrance into the composition. The aromatic cosmetic may be a fragrance (natural or synthetic), an essential oil, or resin oil. Due to the high solvent potential of HFE, the proportions for dilution are not critical. The final mixture may be sprayed, and the scent of the fragrance remains present while the perfluorinated HFE evaporates quickly. However, the incorporation of 10-90% by weight HFE in water is not disclosed.

U.S. Pat. No. 5,304,334 to Lahanas et al. (Lahanas is the inventor of the present application) teaches a method of preparing a stable multiphase composition comprising a continuous phase of silicone fluid and independent polydisperse phases of water, gel, oil, or liquid crystal and perfluoropolyether. The perfluoroolyether is dispersed in the multiphase compistion at 0.5 to about 3% by weight. Such multiphase emulsions tailor delivery of a cosmetic. Each phase can function as an independent delivery system.

U.S. Pat. No. 5,733,526 to Trevino et al. discloses a hydrocarbon oil/fluorochemical preparation, which comprises of a fluorophilic dispersing agent or fluorosurfactant, and may be in the form of a multiple emulsion comprising a polar liquid continuous phase. A bioactive agent may be combined with the polar continuous liquid phase. The dispersing agent can be of the general formula R_{F}—L—R_{H} where R_{F} is a fluorinated alkane of 2 to 10 carbon atoms, L is a linkage unit comprising an oxygen atom, and R_{H} is a linear alkane, branched or cyclic, of 2 to 16 carbon atoms.

Trevino '526, however, only teaches fluorophilic dispersing agents for assisting in dispersing fluorochemicals. These dispersions are anhydrous organic, although they can later be emulsified in water. Nevertheless, the incorporation of 10-90% HFE by weight into water to form a biliquid foam is not taught. Furthermore, the fluorophilic dispersing agents solubilizes hydrocarbon oils, but fragrance oils are not mentioned.

U.S. Pat. No. 6,113,919 to Reiss et al. teaches a fluorocarbon emulsion comprising a fluorophilic/hydrophilic compound which can have the formula R_{F}—W—R_{H} where R_{F} is a linear fluorocarbon group, R_{H} is a linear, branched or cyclic, hydrocarbon group, and W is an oxygen atom (—O—), sulphur atom (—S—), or various fluorinated structures. Use of such a compound provides stable particle size and efficient emulsificaton. Such emulsions are suitable for biomedical use because of the stable particle size and the numerous uses of fluorocabons as oxygen carriers, blood substitutes, and therapeutic and diagnostic agents. The Reiss et al. '919 patent also mentions that the emulsion can be used in cosmetics as well as many other pharmaceutical, biological, and medical arenas.

The fluorophilic/hydrophilic compound in Reiss '919, however, is only a co-solvent or auxillary emulsifier used to couple an oil phase with a fluorocarbon for emulsifying perfluorinated compounds. A biliquid foam of HFE in water is not disclosed.

U.S. Pat. No. 6,165,479 to Wheeler teaches a stable dispersion comprising an oil-based liquid foam and aqueous gel which is suitable for use in the cosmetic industry. The biliquid foam is comprised of a dispersion of oil droplets in an aqueous medium stabilized by only a small amount of surfactant. Although Wheeler '479 discloses a biliquid foam of oil in water in general, it does not teach the incorporation of 10-90% HFE by weight in water specifically. A biliquid foam of volatile HFE in aqueous gel yields a product that dries extremely quickly, has a unique feel, and can be used to deliver fragrances or other active agents.

U.S. Pat. No. 5,965,659 to Kubo et al. discloses a surface treatment composition prepared by emulsifying a fluorine containing oil in water in the presence of a polymeric surfactant of a graft copolymer having a fluorine containing segment. Since fluorine containing oils such as perfluoropolyether require a fluorine-containing solvent for dilution, a graft polymer having a fluorine containing segment may be highly effective in producing the fluorine containing oil in water emulsion. In the graft copolymer, the fluorine containing segment comprises a polymer of a fluorine containing monomer, which preferably comprises a perfluoropolyether group.

### SUMMARY OF THE INVENTION

It is an object of the present invention to provide a safe, non-toxic, non-reactive, and odorless delivery system for cosmetics, active agents, and fragrances in particular, which feels unique to the skin and enhances the aroma of fragrances.

It is another object of the present invention to provide a carrier for active agents that does not result in the absorption of preservatives or the reduction of the concentration of the active agents.

It is a further object of the present invention to provide a delivery system for cosmetics, active agents, and fragrances in particular, which can be dispersed in oil and water.

Accordingly, a perfluorinated oil-in-water biliquid foam is provided as a vehicle for delivering cosmetics and active agents while producing a refreshing and unique contact with the skin, and enhancing the aroma of fragrances within the foam. The biliquid foam comprises hydrofluoroether (“HFE”), surfactants and co-solvents. The biliquid foam may be made by combining non-ionic surfactants with the solvent HFE, and mixing the HFE and surfactant into an aqueous medium such as water or gel via gradually increasing addition that begins dropwise. Various other auxillary cosmetic and non-cosmetic components such as vitamins, anti-oxidants, stabilizers, thickeners, dyes, moisturizers, and preservatives may be added to the biliquid foam as well.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

FIG. 1 is a graphical representation of a perfluorinated oil-in-water biliquid foam; and

FIG. 2 is a block diagram of the method for making a perfluorinated oil-in-water biliquid foam.

### DESCRIPTION OF THE PREFERRED EMBODIMENTS

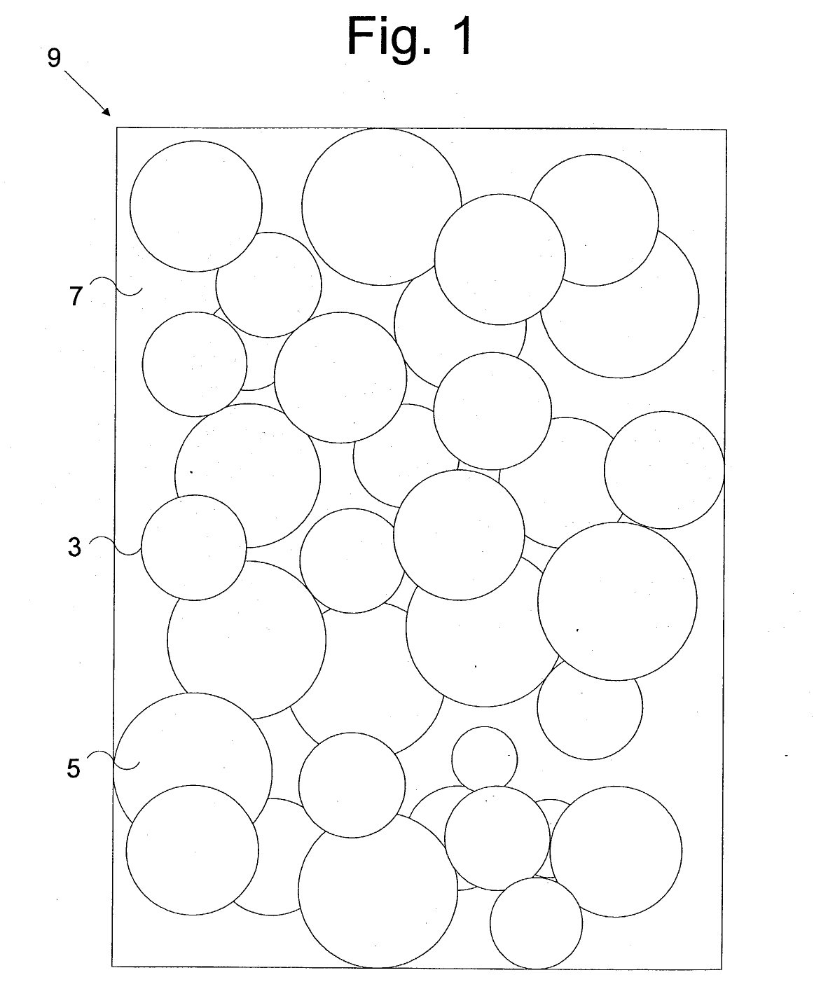

Referring now to the drawings, FIG. 1 shows a multi-phase system in which one or more phases are dispersed in a continuous phase of different composition. The multi-phase system is best described as a concentrated colloidal dispersion, where foam-like cells 3 contain an internal oil phase 5 that is separated from an external aqueous phase 7. The internal oil 5 concentration within the system ranges from 1-99% by weight, and preferably from 10-99% by weight. The system affords an easy method of incorporating oil into water. The internal oil 5 additionally accommodates a necessary reactant and solvent, hydrofluoroether (“HFE”). This type of concentrated colloidal dispersion with foam-like cells separating aqueous and oil into a continuous phase is also known as a biliquid foam, which is generally designated 9.

HFE is preferred for delivering cosmetics, active agents, and in particular, fragrances. The ether linkage within HFE enhances reactivity toward hydroxyl free radicals, and therefore leads to lower atmospheric lifetimes. Therefore, HFE has favorable environmental properties because it does not contain ozone-depleting materials such as CFC. The toxicity of ethers is very low and the ether linkage has a relatively low biological and chemical reactivity. Thus, it is mild on the skin.

HFE is generally an ether containing aliphatic chains with carbon backbones surrounded by hydrogens and fluorines, but is preferred by the present invention to have one aliphatic perfluorinated carbon chain bound to an alkoxy group. HFE is commercially available from 3M as HFE 7100, which is also known as methoxynonafluorobutane, and is designated by the molecular formula C₄F₉OCH₃, having a molecular weight of 250.

HFE 7100 is a clear, odorless, fluid with a high boiling point, high solvency, and low surface tension. It is chemically and thermally stable, inert, non-flammable, and has low toxicity. It is highly volatile and evaporates almost immediately. Typically, it has various uses in industry such as a co-solvent in cleaning heavy oils and greases, an azeotrope in more mild cleaning, and a specialty solvent in dispersion and reaction medium. However, implementation via biliquid foam is necessary because HFE, like most perfluorinated compounds, is extremely difficult to dissolve in water and in most oils.

HFE is particularly valuable to the invention because it enhances the volatility of a fragrance, causing an outstanding “lift,” or cool and unique feeling of evaporation. HFE has no odor and therefore the complementary fragrance may emit an odor without interference from a base odor, such as traditionally found with alcohol based carriers. The HFE containing biliquid foam can be combined with fragrance oil and several other reagents such as pH adjustors, thickeners, emollients, and anti-freeze compounds. When the foam-like cell 3 is released and touches skin, it bursts and the fragrance reacts with the HFE, causing a strong, yet pleasant odor.

HFE is a poor solvent and not generally soluble in water and most oils, however. Nevertheless, it has been determined that HFE is sufficiently soluble in the combination of the volatile, non-polar organic solvent isododecane, and the volatile silicone solvent cyclomethicone.

Preferred volatile nonpolar solvents include branched chain hydrocarbons. Examples of suitable branched chain hydrocarbons include Permethyl 99A (isododecane), Permethyl 102A (isoeicosane), Permethyl 101A (isohexadecane), and combinations thereof. The Permethyl series are available from Preperse, Inc., South Plainfield, N.J., U.S.A.

Other suitable silicone co-solvents may include cyclic, linear or branched chain silicones. Suitable silicones for use herein include, but are not limited to, Cyclomethicone D-5 (commercially available from G. E. Silicones); Dow Corning 344, and Dow Corning 345 (commercially available from Dow Corning Corp.);

GE7207, GE7158 and Silicone Fluids SF-1202 and SF-1173 (available from General Electric Co.)

0038 Non-ionic surfactants are also provided in the oil phase 5 to stabilize the foam-like cells 3 once they are formed. These oil-soluble surfactants are preferably fatty acid ethoxylates and fatty alcohol ethoxylates. In particular, oleth-2 and laureth-4 have been determined as most successful in stabilizing foam-cell 3 formation and are preferred non-ionic surfactants.

Additionally, fragrance oils or oils for other cosmetic purposes may be included in the oil phase mixture as well. Generally, fragrance extracts in the form of aromatic compounds can be dissolved in oil.

The water phase preferably includes deionized water and anionic or hydrophilic surfactants. Sodium Laureth Sulfate (a.k.a., Sodium Lauryl Ether Sulfate) is one anionic surfactant commonly used in cosmetics, which can form a base foam in water and assist in the formation of foam-like cells 3 when the oil phase comes in contact with the water phase. Sodium Laureth Sulfate is commercially available from Chemron under the trade name SulfoChem ES-2.

In alternative embodiments of the invention, and in particular, cosmetic embodiments, the water phase may further include other aqueous-based cosmetics and auxiliary solvents. The water phase may include for example, the thickener and gelling agent Hypan SA-100 and other thickeners such as Carbopol and Natrosol, Triethanolamine (TEA) for pH adjustment and neutralization, 1,3-Butylene Glycol for preventing freezing, the shine controlling agent Gransil Silicone, and various other moisturizers, emollients, humectants and preservatives.

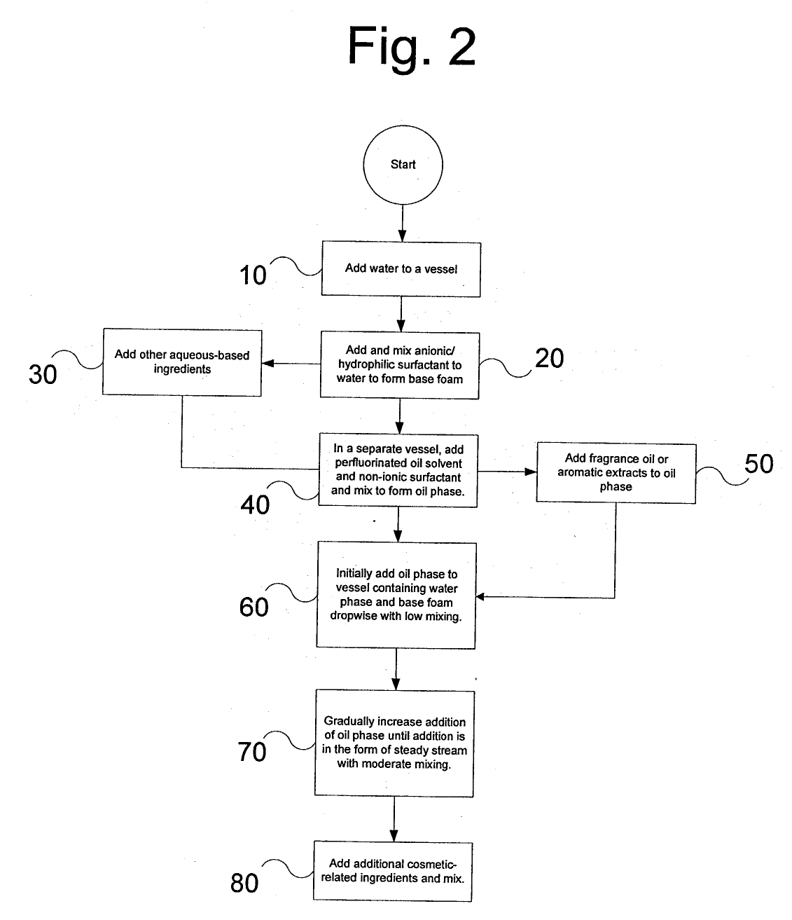

As shown in FIG. 2, the method for making a perfluorinated oil-in-water biliquid foam primarily involves the mixture of a perfluorinated oil phase with a water phase. The water phase is formed by the mixture of water with an anionic or hydrophilic surfactant which forms a base foam (Steps 10-20). Optionally, other water soluble cosmetic ingredients may be added to the water phase (Step 30). The oil phase is made from the combination of the perfluorinated oil, a non-ionic surfactant, and optionally, fragrance oil (Steps 40-50).

The oil phase is added dropwise to the aqueous mixture and base foam with gradual rate increase and low to moderate mixing (Steps 60-70). The foam-like cells 3 are formed with continuing contact between the oil phase and water phase surfaces. Cosmetics can be incorporated in the water and oil phases or added separately into the biliquid foam with additional mixing (Step 80). The formation of the biliquid foam and the addition of cosmetic ingredients is recited in more detail in the exemplary embodiments below.

### EXAMPLE 1

The oil phase preferably comprises HFE 7100, dissolved with isododecane (Permethyl 99A) and cyclomethicone (Dow Corning 345), and mixed with the non-ionic surfactant oleth-2 in a vessel according to the following solute percent by mass per total weight of the solution (% W/W or percent by mass) shown in Table 1 below:

TABLE 1

| Ingredient | % W/W |
|---|---|
| HFE 7100 | 60 |
| Permethyl 99A | 20.2 |
| Dow Corning 345 Fluid | 4.50 |
| Fragrance Oil | 4.40 |
| Oleth-2 | 0.9 |

The oil phase components are mixed until an almost clear solution is obtained. The fragrance oil is not necessary to form the biliquid foam, but is preferred in cosmetic applications. The oil phase may also optionally include various aromatic extracts, such as for example, aromatic orange extract, aromatic ginger root extract, aromatic coriander extract, and aromatic spearmint leaf extract.

In a separate vessel, the aqueous phase is mixed according to Table 2 below:

TABLE 2

| Ingredient | % W/W |
|---|---|
| 1:3 Butylene Glycol | 3.14 |
| 1.68% Sodium Laureth Sulfate | 5.95 |
| Deionized Water | 0.91 |

The aqueous phase solution is mixed with a paddle mixer at about 180-200 rpm. The paddle mixer is placed just at the surface of the water and the aqueous phase is mixed at a rate sufficient to push the water radially against the sides of the vessel. A base biliquid foam becomes visible on the surface due to the surfactant sodium laureth sulfate reacting with water.

The oil phase solution is then added to the vessel containing the aqueous phase at a dropwise rate, gradually increasing to a slow stream. The paddle height is adjusted as the level in the vessel rises to touch the surface of the liquid. As the addition progresses and a dispersion is formed, the rate of addition of the oil phase is increased, with commensurate increase in paddle height. When all of the oil phase has been transferred, the paddle is immersed to ensure uniformity of the phase. The final mixture is mixed for an additional 10 minute, completing the formation of the perfluorinated oil-in-water biliquid foam.

Additionally, various cosmetic-related materials can be combined with the biliquid foam mixture, as shown in Table 3 below:

TABLE 3

| Ingredients | % W/W |
|---|---|
| 1:3 Butylene Glycol | 7.83 |
| Germall Plus | 0.25 |
| 1.5% Hypan | 10.00 |
| Deionized water | 2.07 |
| Caruba Extract Blend | 0.10 |
| Gransil | 3.00 |
| 2% Natrasol | 7.50 |
| Biliquid Foam | 69.25 |

The 1.5% Hypan Solution is made as follows. First, 96.7% by mass deionized water is added into a tank capable of heating and cooling and equipped with propeller agitation. The water is heated to 80-85 degrees centigrade. Hypan SA-100 is weighed out to 1.5% by mass and is slowly added and mixed with the deionized water for 30 minutes with moderate mixing speed.

While maintaining the 80-85 degree temperature, 1.5% by mass 99% TEA is added to the tank with moderate mixing at about 700 rpm clockwise initially. Then mixing is increased to 1400 rpm as the mixture thickens. Finally, mixing rate is decreased to 1200 rpm after an additional 5 minutes. The mixture is cooled to 35-40 degrees centigrade. Germall Plus Powder, at about 0.3% by mass is added to the mixture until the mixture is homogenous.

The 2.0% Natrosol can be made by moderately mixing 2.0% by mass Natrosol 250 with 97.7% by mass deionized water in a tank or vessel with propeller agitation, and then heating the mixture to 45-50 degrees centigrade. After 15-20 minutes of mixing, or after the batch turns clear and homogenous with no visible lumps. Then the batch is cooled to 30-35 degrees centigrade. Finally, 0.3% by mass Gernall Plus Powder is added and mixed until the 2.0% Natrosol solution is homogenous.

The 1:3 Butylene Glycol, Germall Plus, 1.5% Hypan solution, deionized water, and Caruba Extract Blend are weighed and combined into a vessel.

The mixture is homogenized using a Silverson mixer at 350-500 rpm for 15 minutes or until the batch is smooth and lump free. Gransil is mixed in for 15 minutes or until the batch is smooth and lump free. A side sweep mixing blade is then inserted and the 2% Natrosol solution is mixed in at 75 rpm for 15 minutes or until the batch is smooth and lump free. Then the premixed biliquid foam containing HFE is mixed in at 50 rpm until the batch is smooth and homogenous.

### EXAMPLE 2

Another preferred embodiment of the HFE containing Biliquid Foam is shown in Table 4 below:

TABLE 4

| Ingredients | % W/W |
|---|---|
| HFE 7100 | 79.10 |
| Dow Corning 345 | 10.00 |
| Laureth-4 | 0.90 |
| Deionized Water | 9.90 |
| Sodium Laureth Sulfate | 0.10 |

The resulting biliquid foam can then be used with the following exemplary cosmetic mixture shown in Table 5:

TABLE 5

| Ingredients | % W/W |
|---|---|
| Biliquid Foam | 74.00 |
| Hypan SA-100 | 0.10 |
| 99% TEA | 0.10 |
| Deionized Water | 9.80 |
| 1,3 Butylene Glycol | 10.00 |
| Gransil | 3.00 |
| Fragrance Oil | 3.00 |

The Hypan is dissolved in water at 80 degrees centigrade with rapid mixing. TEA is added and the mixture is cooled to room temperature. The fragrance oil is stirred into the biliquid foam and the resulting mixture is added to the Hypan/Water/TEA mixture with stirring. The 1,3 Butylene Glycol and Gransil are added with mixing and the product is homogenized under a Silverson homogenizer.

### EXAMPLE 3:

An alternative cosmetic embodiment may comprise the following ingredients:

TABLE 6

| Ingredient | % W/W | Comments |
|---|---|---|
| Batch #1 | | |
| Deionized Water | 14.30 | |
| Carbopol Ultrez-10 | 0.40 | Thickener |
| Natrosol 250 HHR | 0.20 | Thickener |
| Glycerine | 3.00 | Humectant, |
| | | Emollient |
| 1,3 Butylene Glycol | 5.00 | Emolient, Anti- |
| | | freezing agent |
| Actiglide BBW | 1.50 | Moisturizer, |
| | | Skin |
| | | Conditioner |
| Fucogel | 3.00 | Moisturizer, |
| | | Skin |
| | | Conditioner |
| TEA | 0.20 | pH control |
| Disodium EDTA | 0.10 | Chelating Agent |
| Batch #2 | | |
| Plantaren 2000 (1% aq.) | 4.75 | Nonionic |
| | | surfactant |
| Gransil | 20.00 | Emollient |
| Dow Corning 556 | 2.00 | Emollient |
| Simulgel EG | 0.30 | Thickener |
| Laureth 4 | 0.45 | Nonionic |
| | | surfactant |
| Panalane L-14E | 0.50 | Emollient |
| Batch #3 | | |
| Biliquid Foam w/HFE | 40.00 | |
| Fragrance Oil | 3.00 | |
| Germaben II | 1.00 | Preservative |

In the main vessel, deionized water, Carbopol, Glycerine, Butylene Glycol,, Actiglide BBW, Fucogel, TEA, and Disodium EDTA are combined and mixed until uniform.

In a separate vessel, a base biliquid foam is prepared with Plantaren 2000 solution. Separately, Gransil, Dow Corning 556, Simulgel EG, Laureth 4, and Panalane L-14E are combined, and added slowly to the Plantaren 2000 solution with a paddle mixer on the surface of the liquid to form the base biliquid foam as described in Example 1. The base biliquid foam is then added to the main vessel.

An HFE containing biliquid foam is made by the same ingredients and method as shown in Example 2. Fragrance oil is stirred into the HFE containing biliquid foam until it becomes uniform. The biliquid foam is made in a separate vessel from the main vessel. The resulting oil phase mixture is added to the main aqueous vessel in the manner described in example 1. The preservative Germaben II may be added separately.

While preferred embodiments of the invention have been shown and described in detail to illustrate the application of the principles of the invention, it will be understood that the invention may be embodied otherwise without departing from such principles.

What is claimed is:

1. A perflourinated oil-in-water dispersion in which an oil-based medium is physically separated from an aqueous medium such that a cosmetic or active agent can be carried within the oil-based medium and remain water-free, comprising:
a plurality of foam cells dispersed in said aqueous medium;
a perfluorinated non-toxic and oil-soluble compound contained within said foam cells;
an anionic or hydrophilic emulsifier for forming said foam cells by interacting with said aqueous medium; and
a non-ionic emulsifier for stabilizing said foam cells after formation.

2. A perflourinated oil-in-water dispersion according to claim 1, wherein said perflourinated compound is hydrofluoroether.

3. A perflourinated oil-in-water dispersion according to claim 2, further comprising isododecane and cyclomethicone as co-solvents for dissolving hydrofluoroether.

4. A perflourinated oil-in-water dispersion according to claim 1, wherein said perfluorinated compound is in the range of 10-90% by weight and said aqueous medium is in the range of 1-9%.

5. A perflourinated oil-in-water dispersion according to claim 1, wherein said perfluorinated compound is in the range of 1-99% by weight and said aqueous medium is in the range of 0.1-0.9%.

6. A perflourinated oil-in-water dispersion according to claim 1, wherein said anionic emulsifier is sodium laureth sulfate.

7. A perflourinated oil-in-water dispersion according to claim 1, wherein said nonionic emulsifier is a member of the group consisting of fatty acid ethoxylates and fatty alcohol ethoxylates.

8. A perflourinated oil-in-water dispersion according to claim 1, wherein said cosmetic is a lotion or gel.

9. A perflourinated oil-in-water dispersion according to claim 1, wherein said cosmetic has a fragrance with an aroma that is enhanced when the fragrance is conveyed to skin within said foam cell.

10. A method for making a A perflourinated oil-in-water dispersion, comprising:
mixing an anionic or hydrophilic emulsifier with water to produce a base foam with cells;
mixing a perfluorinated solvent with a non-ionic emulsifier; and
dropwise adding said perfluorinated solvent and non-ionic emulsifier mixture to said base foam with cells with agitation and gradually increasing the rate of said addition to a stream to increase the amount of perfluorinated oil surfaces interacting with aqueous surfaces to produce a biliquid foam containing oil-based foam cells in an aqueous medium.

11. A method for making a perflourinated oil-in-water dispersion according to claim 10, wherein said perflourinated compound is hydrofluoroether.

12. A method for making a perflourinated oil-in-water dispersion according to claim 11, further comprising dissolving hydrofluoroether with a co-solvent.

13. A method for making a perflourinated oil-in-water dispersion according to claim 12, wherein said co-solvent is a mixture of isododecane and cyclomethicone.

14. A method for making a perflourinated oil-in-water dispersion according to claim 10, wherein said perfluorinated compound is in the range of 10-90% by weight and said water is in the range of 1-9%.

15. A method for making a perflourinated oil-in-water dispersion according to claim 10, wherein said perfluorinated compound is in the range of 1-99% by weight and said water is in the range of 0.1-0.9%.

16. A method for making a perflourinated oil-in-water dispersion according to claim 10, wherein said anionic emulsifier is sodium laureth sulfate.

17. A method for making a perflourinated oil-in-water dispersion according to claim 10, wherein said nonionic emulsifier is a member of the group consisting of fatty acid ethoxylates and fatty alcohol ethoxylates.

18. A method for making a perflourinated oil-in-water dispersion perflourinated oil-in-water dispersion according to claim 10, further comprising the addition of a thickener to the water for producing an aqueous gel.

19. A method for making a perflourinated oil-in-water dispersion perflourinated according to claim 10, further comprising the addition of aromatic extracts and fragrance oils to the perfluorinated solvent and non-ionic emulsifier mixture before it is combined with the base foam.
